(19) United States
(12) Patent Application Publication
(10) Pub. No.: US 2010/0010262 A1
(43) Pub. Date: Jan. 14, 2010

(54) COMPOSITIONS AND METHOD FOR MAKING THEREOF

(75) Inventors: Gary Charles Davis, Albany, NY (US); Kathryn Longley, Saratoga Springs, NY (US); Moitreyee Sinha, Niskayuna, NY (US); Christoph Georg Erben, Clifton Park, NY (US); Sumeet Jain, Niskayuna, NY (US)

Correspondence Address: GENERAL ELECTRIC COMPANY;GLOBAL RESEARCH, ONE RESEARCH CIRCLE, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 12/169,655

(22) Filed: Jul. 9, 2008

(51) Int. Cl. C07C 249/00

(52) U.S. Cl. 564/248

(57) ABSTRACT

A composition has a structure as shown in formula I:

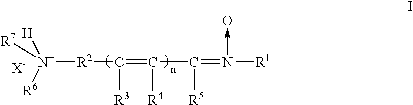

R¹ and R² are independently at each occurrence an aliphatic radical having from 1 to about 10 carbons, a cycloaliphatic radical having from about 3 to about 10 carbons, or an aromatic radical having from about 3 to about 12 carbons; R³, R⁴, and R⁵ are independently at each occurrence a hydrogen atom, an aliphatic radical having from 1 to about 10 carbons, a cycloaliphatic radical having from about 3 to about 10 carbons, or an aromatic radical having from about 3 to about 12 carbons; R⁶ and R⁷ are independently at each occurrence a hydrogen atom or an aliphatic radical having from 1 to about 6 carbons; X is a halogen; and “n” is an integer having a value of from 0 to about 4.

### BACKGROUND

1. Technical Field

The invention includes embodiments that may relate to compositions including protonated nitrone dyes. The invention includes embodiments that may relate to a holographic recording medium. The invention includes embodiments that may relate to a method for making and using a holographic recording medium.

2. Discussion of Art

Holographic recording is the storage of information in the form of holograms. The information can be stored in different forms including binary data, images, bar-codes, and gratings. Holograms are images of three-dimensional interference pattern. These patterns may be created by the intersection of two beams of light in a photosensitive medium. A difference of volume holographic recording relative to surface-based storage formats is that a large number of holograms may be stored in an overlapping manner in the same volume of the photosensitive medium using a multiplexing technique. This multiplexing technique may vary the signal and/or reference beam angle, wavelength, or medium position. However, an impediment towards the realization of holographic recording as a viable technique has been the development of a suitable recording medium.

Recent holographic recording materials work has led to the development of dye-doped data polymeric materials. The sensitivity of a dye-doped data storage material may depend on the concentration of the dye, the dye's absorption cross-section at the recording wavelength, the quantum efficiency of the photochemical transition, and the index change of the dye molecule for a unit dye density. However, as the product of dye concentration and the absorption cross-section increases, the storage medium (for example, an optical data storage disc) may become opaque, which may complicate both recording and readout.

It may be desirable to have compositions, articles, or a holographic recording medium that has characteristics and properties that differ from those currently available.

### Brief Description

In one embodiment, a composition is provided that has a structure as shown in formula I:

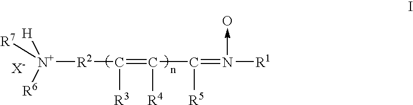

wherein R¹ and R² are independently at each occurrence an aliphatic radical having from 1 to about 10 carbons, a cycloaliphatic radical having from about 3 to about 10 carbons, or an aromatic radical having from about 3 to about 12 carbons; R³, R⁴, and R⁵ are independently at each occurrence a hydrogen atom, an aliphatic radical having from 1 to about 10 carbons, a cycloaliphatic radical having from about 3 to about 10 carbons, or an aromatic radical having from about 3 to about 12 carbons; R⁶ and R⁷ are independently at each occurrence a hydrogen atom or an aliphatic radical having from 1 to about 6 carbons; X is a halogen; and “n” is an integer having a value of from 0 to about 4.

In one embodiment, an article is provided that includes a composition having a structure as shown in formula I.

In one embodiment, a composition is provided that has a structure as shown in formula IX.

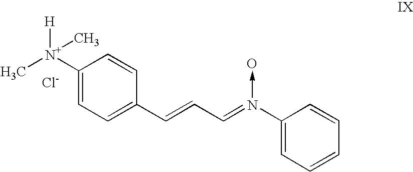

In one embodiment, an article is provided that includes a composition having a structure as shown in formula IX.

In one embodiment, a composition having a structure as shown in formula X is provided.

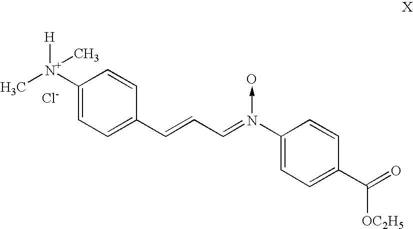

In one embodiment, an article is provided that includes a composition having a structure as shown in formula X.

In one embodiment, a method prepares the composition having the structure as shown in formula I. In one embodiment, a method prepares the composition having a structure as shown in formula IX. In one embodiment, a method prepares the composition having a structure as shown in formula X.

### BRIEF DESCRIPTION OF FIGURES

FIG. 1 shows a change in absorbance of a photochemically active dye according to an embodiment of the invention

FIG. 2 shows a change in absorbance of a photochemically active dye according to an embodiment of the invention.

FIG. 3 shows a change in refractive index of a photochemically active dye according to an embodiment of the invention.

FIG. 4 shows a refractive index change of a photosensitive material according to an embodiment of the invention.

FIG. 5 shows a diffraction efficiency change of a photosensitive material according to an embodiment of the invention.

FIG. 6 shows a hologram erasure measurement of an article according to an embodiment of the invention.

### DETAILED DESCRIPTION

The invention includes embodiments that may relate to compositions including protonated nitrone dyes. The invention includes embodiments that may relate to a holographic recording medium. The invention includes embodiments that may relate to a method for making and using the compositions to form an article, such as the holographic recording medium.

In one embodiment, a composition has a structure as shown in formula I:

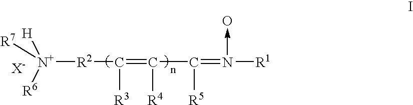

and R¹ and R² can be independently at each occurrence an aliphatic radical having from 1 to about 10 carbons, a cycloaliphatic radical having from about 3 to about 10 carbons, or an aromatic radical having from about 3 to about 12 carbons. R³, R⁴, and R⁵ are independently at each occurrence a hydrogen atom, an aliphatic radical having from 1 to about 10 carbons, a cycloaliphatic radical having from about 3 to about 10 carbons, or an aromatic radical having from about 3 to about 12 carbons. R⁶ and R⁷ are independently at each occurrence a hydrogen atom or an aliphatic radical having from 1 to about 6 carbons. X is a halogen; and, “n” is an integer having a value of from 0 to about 4. Selection of moieties may affect one or more performance characteristics of the resultant material, and may require processing changes to achieve the resultant material, or to use the resultant material.

In one embodiment, R¹ is an aromatic radical having from about 5 to about 12 carbons; R² is an aromatic radical having from about 5 to about 12 carbons; R³, R⁴, and R⁵ are independently at each occurrence a hydrogen atom, an aliphatic radical having from 1 to about 10 carbons, a cycloaliphatic radical having from about 3 to about 10 carbons, or an aromatic radical having from about 3 to about 12 carbons. In one embodiment, X is chlorine. In one embodiment, X is bromine. In one embodiment, X is iodine.

In one embodiment, R¹ is an aromatic radical having from about 6 to about 10 carbons; R² is an aromatic radical having from about 6 to about 10 carbons; R³, R⁴, and R⁵ are independently at each occurrence a hydrogen atom, an aliphatic radical having from 1 to about 5 carbons, a cycloaliphatic radical having from about 4 to about 8 carbons, or an aromatic radical having from about 6 to about 10 carbons; and “n” is an integer having a value of from 1 to 3.

In one embodiment, R¹ comprises at least one electron withdrawing substituent having a structure selected from the group consisting of formulae;

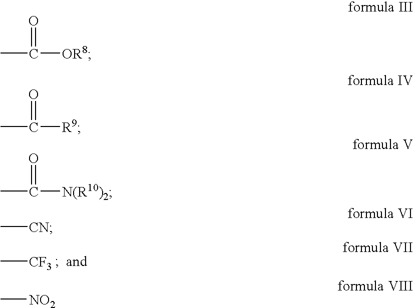

wherein R⁸, R⁹, and R¹⁰ are each independently at each occurrence an aliphatic radical having 1 to 10 carbons, a cycloaliphatic radical having about 3 to 10 carbons, and an aromatic radical having from about 3 to 10 carbons.

As used herein, the term “aromatic radical” refers to an array of atoms having a valence of at least one including at least one aromatic group. The array of atoms having a valence of at least one including at least one aromatic group may include heteroatoms such as nitrogen, sulfur, selenium, silicon and oxygen, or may be composed exclusively of carbon and hydrogen. As used herein, the term “aromatic radical” includes but is not limited to phenyl, pyridyl, furanyl, thienyl, naphthyl, phenylene, and biphenyl radicals. As noted, the aromatic radical contains at least one aromatic group. The aromatic group is invariably a cyclic structure having 4n+2 “delocalized” electrons where “n” is an integer equal to 1 or greater, as illustrated by phenyl groups (n=1), thienyl groups (n=1), furanyl groups (n=1), naphthyl groups (n=2), azulenyl groups (n=2), anthraceneyl groups (n=3) and the like. The aromatic radical may also include nonaromatic components. For example, a benzyl group is an aromatic radical that includes a phenyl ring (the aromatic group) and a methylene group (the nonaromatic component). Similarly a tetrahydronaphthyl radical is an aromatic radical including an aromatic group (C₆H₃) fused to a nonaromatic component —(CH₂)₄—. For convenience, the term “aromatic radical” is defined herein to encompass a wide range of functional groups such as alkyl groups, alkenyl groups, alkynyl groups, haloalkyl groups, haloaromatic groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylphenyl radical is a C₇ aromatic radical including a methyl group, the methyl group being a functional group which is an alkyl group. Similarly, the 2-nitrophenyl group is a C₆ aromatic radical including a nitro group, the nitro group being a functional group. Aromatic radicals include halogenated aromatic radicals such as 4-trifluoro methyl phenyl, hexafluoro isopropylidene bis (4-phen-1-yloxy) (i.e., —OPhC(CF₃)₂PhO—); 4-chloromethylphen-1-yl, 3-trifluorovinyl-2-thienyl, 3-trichloro methylphen-1-yl (i.e., 3-CCl₃Ph-); 4-(3-bromoprop-1-yl)phen-1-yl (i.e., 4-BrCH₂CH₂CH₂Ph-); and the like. Further examples of aromatic radicals include 4-allyloxyphen-1-oxy; 4-aminophen-1-yl (i.e., 4-H₂NPh-); 3-aminocarbonylphen-1-yl (i.e., NH₂COPh-); 4-benzoylphen-1-yl; dicyano methylidene bis (4-phen-1-yl oxy) (i.e., —OPhC(CN)₂PhO—); 3-methylphen-1-yl, methylene bis (4-phen-1-yl oxy) (i.e., —OPhCH₂PhO—); 2-ethylphen-1-yl, phenyl ethenyl, 3-formyl-2-thienyl, 2-hexyl-5-furanyl; hexamethylene-1,6-bis(4-phen-1-yloxy) (i.e., —OPh(CH₂)₆PhO—); 4-hydroxy methylphen-1-yl (i.e., 4-HOCH₂Ph-); 4-mercapto methylphen-1-yl (i.e., 4-HSCH₂Ph-); 4-methylthiophen-1-yl (i.e., 4-CH₃SPh-); 3-methoxyphen-1-yl; 2-methoxy carbonyl phen-1-yloxy (e.g., methyl salicyl); 2-nitromethylphen-1-yl (i.e., 2-NO₂CH₂Ph); 3-trimethylsilylphen-1-yl; 4-t-butyl dimethylsilylphenl-1-yl; 4-vinylphen-1-yl; vinylidene bis (phenyl); and the like. The term “a C₃-C₁₀ aromatic radical” includes aromatic radicals containing at least three but no more than 10 carbon atoms. The aromatic radical 1-imidazolyl (C₃H₂N₂—) represents a C₃ aromatic radical. The benzyl radical (C₇H₇—) represents a C₇ aromatic radical.

As used herein, the term “cycloaliphatic radical” refers to a radical having a valence of at least one, and including an array of atoms which is cyclic but which is not aromatic. As defined herein a “cycloaliphatic radical” does not contain an aromatic group. A “cycloaliphatic radical” may include one or more noncyclic components. For example, a cyclohexylmethyl group (C₆H₁₁CH₂—) is a cycloaliphatic radical that includes a cyclohexyl ring (the array of atoms which is cyclic but which is not aromatic) and a methylene group (the noncyclic component). The cycloaliphatic radical may include heteroatoms such as nitrogen, sulfur, selenium, silicon and oxygen, or may be composed exclusively of carbon and hydrogen. For convenience, the term “cycloaliphatic radical” is defined herein to encompass a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methyl cyclopent-1-yl radical is a C₆ cycloaliphatic radical including a methyl group, the methyl group being a functional alkyl group. Similarly, the 2-nitrocyclobut-1-yl radical is a C₄ cycloaliphatic radical including a nitro group, the nitro group being a functional group. A cycloaliphatic radical may include one or more halogen atoms which may be the same or different from each other. Halogen atoms include, for example; fluorine, chlorine, bromine, and iodine. Cycloaliphatic radicals including one or more halogen atoms include 2-trifluoro methylcyclohex-1-yl; 4-bromo difluoro methyl cyclo oct-1-yl; 2-chloro difluoro methylcyclohex-1-yl; hexafluoro isopropylidene-2,2-bis(cyclohex-4-yl) (i.e., —C₆H₁₀C(CF₃)₂ C₆H₁₀—); 2-chloro methylcyclohex-1-yl; 3-difluoro methylene cyclohex-1-yl; 4-trichloro methyl cyclohex-1-yloxy; 4-bromo dichloro methylcyclohex-1-yl thio; 2-bromo ethyl cyclopent-1-yl; 2-bromo propyl cyclo hex-1-yloxy (e.g., CH₃CHBrCH₂C₆H₁₀O—); and the like. Further examples of cycloaliphatic radicals include 4-allyl oxycyclo hex-1-yl; 4-amino cyclohex-1-yl (i.e., H₂NC₆H₁₀—); 4-amino carbonyl cyclopent-1-yl (i.e., NH₂COC₅H₈—); 4-acetyl oxycyclo hex-1-yl; 2,2-dicyano isopropylidene bis(cyclohex-4-yloxy) (i.e., —OC₆H₁₀C(CN)₂C₆H₁₀O—); 3-methyl cyclohex-1-yl; methylene bis (cyclohex-4-yloxy) (i.e., —OC₆H₁₀CH₂C₆H₁₀O—); 1-ethyl cyclobut-1-yl; cyclo propyl ethenyl, 3-formyl-2-terahydrofuranyl; 2-hexyl-5-tetrahydrofuranyl; hexamethylene-1,6-bis(cyclohex-4-yloxy) (i.e., —OC₆H₁₀(CH₂)₆C₆H₁₀O—); 4-hydroxy methylcyclohex-1-yl (i.e., 4-HOCH₂C₆H₁₀—), 4-mercapto methyl cyclohex-1-yl (i.e., 4-HSCH₂C₆H₁₀13 ), 4-methyl thiocyclohex-1-yl (i.e., 4-CH₃SC₆H₁₀—); 4-methoxy cyclohex-1-yl, 2-methoxy carbonyl cyclohex-1-yloxy (2-CH₃OCOC₆H₁₀O—), 4-nitro methyl cyclohex-1-yl (i.e., NO₂CH₂C₆H₁₀); 3-trimethyl silyl cyclohex-1-yl; 2-t-butyl dimethylsilylcyclopent-1-yl; 4-trimethoxy silylethyl cyclohex-1-yl (e.g., (CH₃O)₃SiCH₂CH₂C₆H₁₀—); 4-vinyl cyclohexen-1-yl; vinylidene bis(cyclohexyl), and the like. The term “a C₃-C₁₀ cycloaliphatic radical” includes cycloaliphatic radicals containing at least three but no more than 10 carbon atoms. The cycloaliphatic radical 2-tetrahydrofuranyl (C₄H₇O—) represents a C₄ cycloaliphatic radical. The cyclohexylmethyl radical (C₆H₁₁CH₂—) represents a C₇ cycloaliphatic radical.

As used herein, the term “aliphatic radical” refers to an organic radical having a valence of at least one consisting of a linear or branched array of atoms that is not cyclic. Aliphatic radicals are defined to include at least one carbon atom. The array of atoms including the aliphatic radical may include heteroatoms such as nitrogen, sulfur, silicon, selenium and oxygen or may be composed exclusively of carbon and hydrogen. For convenience, the term “aliphatic radical” is defined herein to encompass, as part of the “linear or branched array of atoms which is not cyclic” a wide range of functional groups such as alkyl groups, alkenyl groups, alkynyl groups, haloalkyl groups, conjugated dienyl groups, alcohol groups, ether groups, aldehyde groups, ketone groups, carboxylic acid groups, acyl groups (for example carboxylic acid derivatives such as esters and amides), amine groups, nitro groups, and the like. For example, the 4-methylpent-1-yl radical is a C₆ aliphatic radical including a methyl group, the methyl group being a functional alkyl group. Similarly, the 4-nitrobut-1-yl group is a C₄ aliphatic radical including a nitro group, the nitro group being a functional group. An aliphatic radical may be a haloalkyl group which includes one or more halogen atoms which may be the same or different. Halogen atoms include, for example; fluorine, chlorine, bromine, and iodine. Aliphatic radicals including one or more halogen atoms include the alkyl halides trifluoromethyl; bromodifluoromethyl; chlorodifluoromethyl; hexafluoroisopropylidene; chloromethyl; difluorovinylidene; trichloromethyl; bromodichloromethyl; bromoethyl; 2-bromotrimethylene (e.g., —CH₂CHBrCH₂—); and the like. Further examples of aliphatic radicals include allyl; aminocarbonyl (i.e., —CONH₂); carbonyl; 2,2-dicyano isopropylidene (i.e., —CH₂C(CN)₂CH₂—); methyl (i.e., —CH₃); methylene (i.e., —CH₂—); ethyl; ethylene; formyl (i.e., —CHO); hexyl; hexamethylene; hydroxymethyl (i.e., —CH₂OH); mercaptomethyl (i.e., —CH₂SH); methylthio (i.e., —SCH₃); methylthiomethyl (i.e., —CH₂SCH₃); methoxy; methoxycarbonyl (i.e., CH₃OCO—); nitromethyl (i.e., —CH₂NO₂); thiocarbonyl; trimethylsilyl (i.e., (CH₃)₃Si—); t-butyldimethylsilyl; 3-trimethyoxysilylpropyl (i.e., (CH₃O)₃SiCH₂CH₂CH₂—); vinyl; vinylidene; and the like. By way of further example, a C₁-C₁₀ aliphatic radical contains at least one but no more than 10 carbon atoms. A methyl group (i.e., CH₃—) is an example of a C₁ aliphatic radical. A decyl group (i.e., CH₃(CH₂)₉—) is an example of a C₁₀ aliphatic radical.

In one embodiment, an article includes a composition having a structure as shown in formula I. In one embodiment, the article is a holographic recording medium. Non-limiting examples of the article include optical media storage, biometric access cards, and credit cards.

In one embodiment, the composition having a structure as shown in formula I may be prepared by protonating a composition having a structure as shown in formula II

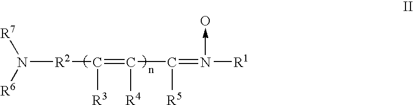

and R¹, R², R³, R⁴, R⁵, R⁶, R⁷, X, and “n” have the same meaning as provided for formula I above.

In one embodiment, a composition having a structure as shown in formula IX is provided.

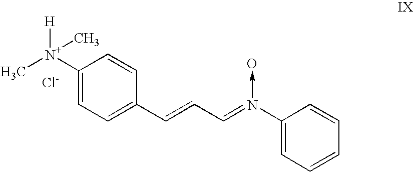

In one embodiment, the composition having a structure as shown in formula IX may be prepared by protonating a composition having a structure as shown in formula XI.

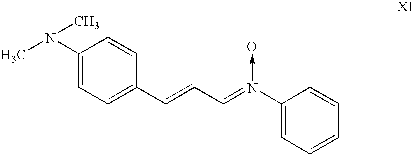

The composition having a structure shown in formula IX may also be referred to as alpha-(4-dimethylaminostyryl)-N-phenyl nitrone hydrochloride. The composition having a structure shown in formula XI may also be referred to as alpha-(4-dimethylaminostyryl)-N-phenyl nitrone. In one embodiment, is provided an article. The article includes a composition having a structure as shown in formula IX and XI.

In one embodiment, a composition having a structure as shown in formula X is provided.

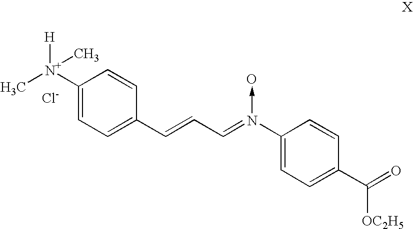

In one embodiment, the composition having a structure as shown in formula X may be prepared by protonating a composition having a structure as shown in formula XII.

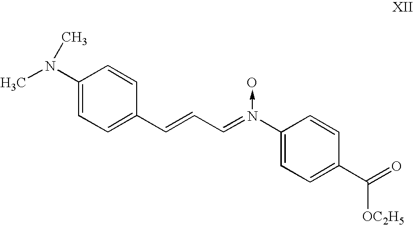

The composition having a structure shown in formula X may also be referred to as alpha-(4-methylaminostyryl)-N-(4-carbethoxyphenyl)nitrone hydrochloride. The composition having a structure shown in formula XII may also be referred to as alpha-(4-methylaminostyryl)-N-(4-carbethoxyphenyl)nitrone. In one embodiment, an article includes a composition having a structure as shown in formula X and XII. Protonating the composition may be achieved by exposing the composition having a structure as shown in formula I to an acid. In one embodiment, the type of acid will be dependent on the type of the dye that needs to be protonated. Non-limiting examples of acids include hydrochloric acid, hydrobromic acid, and hydroiodic acid.

In one embodiment, a holographic recording medium is provided that includes an optically transparent substrate. The optically transparent substrate includes a photochemically active dye, and a protonated form of the photochemically active dye. The protonated form of the photochemically active dye is a composition having a structure as shown in formula I

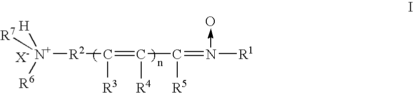

and the photochemically active dye is a composition having a structure as shown in formula II

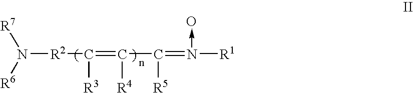

wherein in both formulae I and II, R¹ and R² can independently at each occurrence be an aliphatic radical having from 1 to about 10 carbons, a cycloaliphatic radical having from about 3 to about 10 carbons, or an aromatic radical having from about 3 to about 12 carbons; R³, R⁴, and R⁵ are independently at each occurrence a hydrogen atom, an aliphatic radical having from 1 to about 10 carbons, a cycloaliphatic radical having from about 3 to about 10 carbons, or an aromatic radical having from about 3 to about 12 carbons; R⁶ and R⁷ are independently at each occurrence a hydrogen atom or an aliphatic radical having from 1 to about 6 carbons; X is a halogen; and “n” is an integer having a value of from 0 to about 4.

In one embodiment, the optically transparent substrate has an absorbance of greater than about 0.1 at a wavelength that is in a range of from about 300 nanometers to about 1000 nanometers. In one embodiment, the optically transparent substrate has an absorbance of from about 0.1 to about 5 at a wavelength that is in a range of from about 300 nanometers to about 1000 nanometers. In one embodiment, the optically transparent substrate has an absorbance of from about 0.1 to about 1, from about 1 to about 2, from about 2 to about 3, from about 3 to about 4, and from about 4 to about 5 at a wavelength that is in a range of from about 300 nanometers to about 1000 nanometers. In one embodiment, the optically transparent substrate has an absorbance of greater than about 0.1 at a wavelength that is in range from about 300 nanometers to about 400 nanometers, from about 400 nanometers to about 500 nanometers, from about 500 nanometers to about 600 nanometers, from about 600 nanometers to about 700 nanometers, from about 700 nanometers to about 800 nanometers, from about 800 nanometers to about 900 nanometers, and from about 900 nanometers to about 1000 nanometers.

In one embodiment, the optically transparent substrate may have a diffraction efficiency of greater than about 10 percent. In one embodiment, the optically transparent substrate may have a diffraction efficiency of from about 10 percent to about 50 percent. In one embodiment, the optically transparent substrate may have a diffraction efficiency of from about 10 percent to 30 percent, from about 20 percent to 30 percent, from about 30 percent to about 40 percent, or from about 40 percent to about 50 percent, or greater. The reported diffraction efficiency values are corrected for background absorption and surface reflection.

In one embodiment, the holographic recording medium may have a data storage capacity that is greater than about 1. As defined herein, the phrase data storage capacity relates to the capacity of a holographic recording medium as given by M/#. M/# can be measured as a function of the total number of multiplexed holograms that can be recorded at a volume element of the data storage medium at a given diffraction efficiency. M/# depends upon various parameters, such as the change in refractive index (Δn), the thickness of the medium, and the dye concentration. These terms are described further in this disclosure. The M# is defined as shown in equation 1:

$$M / \# = {\sum }_{i = 1}^{N} \sqrt{{η}_{i}} \qquad Equation 1$$

where ηᵢ is diffraction efficiency of the i^{th} hologram, and N is the number of recorded holograms. The experimental setup for M/# measurement for a test sample at a chosen wavelength, for example, at 532 nanometers or 405 nanometers involves positioning the testing sample on a rotary stage that is controlled by a computer. The rotary stage has a high angular resolution, for example, about 0.0001 degree. An M/# measurement involves two steps: recording and readout. At recording, multiple plane-wave holograms are recorded at the same location on the same sample. A plane wave hologram is a recorded interference pattern produced by a signal beam and a reference beam. The signal and reference beams are coherent to each other. They are both plane-waves that have the same power and beam size, incident at the same location on the sample, and polarized in the same direction. Multiple plane-wave holograms are recorded by rotating the sample. Angular spacing between two adjacent holograms is about 0.2 degree. This spacing is chosen so that their impact to the previously recorded holograms, when multiplexing additional holograms, is minimal and at the same time, the usage of the total capacity of the media is efficient. Recording time for each hologram is generally the same in M/# measurements. At readout, the signal beam is blocked. The diffracted signal is measured using the reference beam and an amplified photo-detector. Diffracted power is measured by rotating the sample across the recording angle range with a step size of about 0.004 degree. The power of the reference beam used for readout may be about 2-3 orders of magnitude smaller than that used at recording. This is to minimize hologram erasure during readout while maintaining a measurable diffracted signal. From the diffracted signal, the multiplexed holograms can be identified from the diffraction peaks at the hologram recording angles. The diffraction efficiency of the i^{th} hologram, ηᵢ, is then calculated by using Equation 2:

$${η}_{i} = \frac{{P}_{i , diffracted}}{{P}_{reference}} \qquad Equation 2$$

where P_{i, diffracted} is the diffracted power of the i^{th} hologram. M/# is then calculated using the diffraction efficiencies of the holograms and Equation 1. Thus, a holographic plane wave characterization system may be used to test the characteristics of the data storage material, especially multiplexed holograms. Further, the characteristics of the data storage material can also be determined by measuring the diffraction efficiency.

As used herein, the term “volume element” means a three dimensional portion of the total volume of an optically transparent substrate or a modified optically transparent substrate. “Optically transparent” refers to a property that allows about 90 percent or more light to propagate through where the light has a determined wavelength in the visible light range. A hologram is a diffraction pattern.

As defined herein, the term “optically readable datum” is made up of one or more volume elements of a first or a modified optically transparent substrate containing a “hologram” of the data to be stored. The refractive index within an individual volume element may be constant throughout the volume element, as in the case of a volume element that has not been exposed to electromagnetic radiation, or in the case of a volume element in which the photochemically active dye has been reacted to the same degree throughout the volume element. Some volume elements that have been exposed to electromagnetic radiation during the holographic data writing process may contain a complex holographic pattern. And, the refractive index within the volume element may vary across the volume element. In instances in which the refractive index within the volume element varies across the volume element, it is convenient to regard the volume element as having an “average refractive index” which may be compared to the refractive index of the corresponding volume element prior to irradiation. Thus, in one embodiment an optically readable datum includes at least one volume element having a refractive index that is different from the corresponding volume element of the optically transparent substrate prior to irradiation. Locally changing the refractive index of the data storage medium in a graded fashion (continuous sinusoidal variations), rather than discrete steps, and then using the induced changes as diffractive optical elements allows data storage.

The capacity to store data as holograms (M/#) may be directly proportional to the ratio of the change in refractive index per unit dye density (Δn/N₀) at the wavelength used for reading the data to the absorption cross section (σ) at a given wavelength used for writing the data as a hologram. The refractive index change per unit dye density is given by the ratio of the difference in refractive index of the volume element before irradiation minus the refractive index of the same volume element after irradiation to the density of the dye molecules. The refractive index change per unit dye density has a unit of (centimeter)³. Thus in an embodiment, the optically readable datum includes at least one volume element wherein the ratio of the change in the refractive index per unit dye density of the at least one volume element to an absorption cross section of the at least one photochemically active dye is at least about 10⁻⁵ expressed in units of centimeter.

Sensitivity (S) is a measure of the diffraction efficiency of a hologram recorded using a certain amount of light fluence (F). The light fluence (F) is given by the product of light intensity (i) and recording time (t). Mathematically, sensitivity may be expressed by Equation 3,

$$S = \frac{\sqrt{η}}{I \cdot t \cdot L} ( cm / J ) \qquad Equation 3$$

wherein “i” is the intensity of the recording beam, “t” is the recording time, L is the thickness of the recording (or data storage) medium (example, disc), and η is the diffraction efficiency. Diffraction efficiency is given by Equation 4,

$$η = {sin}^{2} ( \frac{π \cdot Δ n \cdot L}{λ \cdot cos ( θ )} ) \qquad Equation 4$$

wherein λ is the wavelength of light in the recording medium, θ is the recording angle in the media, and Δn is the refractive index contrast of the grating, which is produced by the recording process, wherein the dye molecule undergoes a photochemical conversion.

The absorption cross section is a measurement of an atom or molecule's ability to absorb light at a specified wavelength, and is measured in square centimeters per molecule. It is generally denoted by σ(λ) and is governed by the Beer-Lambert Law for optically thin samples as shown in Equation 5,

$$σ ( λ ) = ln ( 10 ) \cdot \frac{Absorbance ( λ )}{{N}_{o} \cdot L} ( {cm}^{2} ) \qquad Equation 5$$

wherein N₀ is the concentration in molecules per cubic centimeter, and L is the sample thickness in centimeters.

Quantum efficiency (QE) is a measure of the probability of a photochemical transition for each absorbed photon of a given wavelength. Thus, it gives a measure of the efficiency with which incident light is used to achieve a given photochemical conversion, also called as a bleaching process. QE is given by equation 6,

$$QE = \frac{hc / λ}{σ \cdot {F}_{0}} \qquad Equation 6$$

wherein “h” is the Planck's constant, “c” is the velocity of light, σ(λ) is the absorption cross section at the wavelength λ, and F₀ is the bleaching fluence. The parameter F₀ is given by the product of light intensity (i) and a time constant (τ) that characterizes the bleaching process.

In one embodiment, the photochemically active dye present in the optically transparent substrate is from about 0.1 weight percent to about 20 weight percent. In one embodiment, the photochemically active dye is present in the optically transparent substrate in an amount from about 0.1 weight percent to about 2 weight percent, from about 2 weight percent to about 4 weight percent, from about 4 weight percent to about 6 weight percent, from about 6 weight percent to about 8 weight percent, from about 8 weight percent to about 10 weight percent, from about 10 weight percent to about 12 weight percent, from about 12 weight percent to about 14 weight percent, from about 14 weight percent to about 16 weight percent, from about 16 weight percent to about 18 weight percent, and from about 18 weight percent to about 20 weight percent. As used herein, the term “weight percent” of the dye refers to a ratio of the weight of the dye included in the optically transparent substrate to the total weight of the optically transparent substrate (inclusive of the weight of the dye). For example, 10 weight percent of the dye disposed in an optically transparent substrate implies 10 grams of the dye in 90 grams of the optically transparent substrate. The loading percentage of the dye may be controlled to provide desirable properties based on the characteristics of the dye and the optically transparent substrate.

A photochemically active dye may be described as a dye molecule that has an optical absorption resonance characterized by a center wavelength associated with the maximum absorption and a spectral width (full width at half of the maximum, FWHM) of less than 500 nanometers. In addition, the photochemically active dye molecule may undergo a partial light induced chemical reaction when exposed to light with a wavelength within the absorption range to form at least one photo-product. In various embodiments, this reaction may be a photo-decomposition reaction, such as oxidation, reduction, or bond breaking to form smaller constituents, or a molecular rearrangement, such as for example a sigmatropic rearrangement, or addition reactions including pericyclic cycloadditions. Thus in an embodiment, data storage in the form of holograms may be achieved wherein the photo-product is patterned (for example, in a graded fashion) within the modified optically transparent substrate to provide the at least one optically readable datum.

In one embodiment, the photoproduct of the photochemically active dye having formula II may have a formula as shown below,

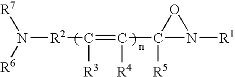

wherein R¹, R², R³, R⁴, and R⁵, R⁶ and R⁷ and X and “n” have the same meanings as provided for formula II.

In one embodiment, the holographic recording medium includes a composition having a structure as shown in formula IX. In one embodiment, the holographic recording medium including a composition having a structure as shown in formula IX may be prepared by exposing an holographic recording medium including a composition having a structure as shown in formula XI to acid, resulting in the holographic recording medium including a composition having a structure as shown in formula IX and formula XI. In one embodiment, the holographic recording medium may include the photo-product of the composition having a structure as shown in formula XI. The photo-product may have a structure as shown in formula XIII.

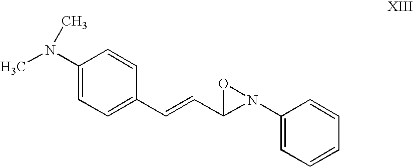

In one embodiment, the holographic recording medium includes a composition having a structure as shown in formula X. In one embodiment, the holographic recording medium including a composition having a structure as shown in formula X may be prepared by exposing an holographic recording medium including a composition having a structure as shown in formula XII to acid, resulting in the holographic recording medium including a composition having a structure as shown in formula X and formula XII. In one embodiment, the holographic recording medium may include the photo-product of the composition having a structure as shown in formula XII. The photo-product may have a structure as shown in formula XIV.

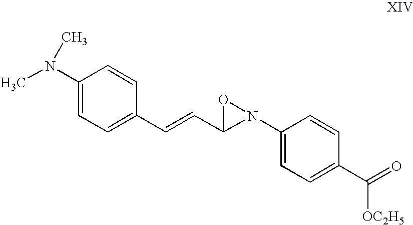

In one embodiment, the optically transparent substrate is greater than about 20 micrometers thick. In one embodiment, the optically transparent substrate is about 20 micrometers to about 50 micrometers thick, about 50 micrometers to about 100 micrometers thick, about 100 micrometers to about 150 micrometers thick, about 150 micrometers to about 200 micrometers thick, about 200 micrometers to about 250 micrometers thick, or about 250 micrometers to about 300 micrometers thick, about 300 micrometers to about 350 micrometers thick, about 350 micrometers to about 400 micrometers thick, about 400 micrometers to about 450 micrometers thick, about 450 micrometers to about 500 micrometers thick, about 500 micrometers to about 550 micrometers thick, about 550 micrometers to about 600 micrometers thick, or greater.

In one embodiment, the optically transparent substrates may include but are not limited to glass, plastic, ink, adhesive, and combinations thereof. Non-limiting examples of glass may include quartz glass and borosilicate glass. Non-limiting examples of plastic may include organic polymers. Suitable organic polymers may include thermoplastic polymers chosen from polyethylene terephthalate, polyethylene naphthalate, polyethersulfone, polycarbonate, polyimide, polyacrylate, polyolefin, and thermoset polymers. In one embodiment, the optically transparent substrate may include a coating of plastic, ink, or adhesives on a substrate such as glass. In one embodiment, the optically transparent substrate may be coated with a reflective coating. For example, if the optically transparent substrate is an optical media such as DVD, a reflective coating may be applied to either one or both the surfaces of the DVD. Examples of reflective coatings include metal coatings such as silver coating.

In one embodiment, the optically transparent substrate used in producing the holographic recording media may include any plastic material having sufficient optical quality, e.g., low scatter, low birefringence, and negligible losses at the wavelengths of interest, to render the data in the holographic recording material readable. Organic polymeric materials, such as for example, oligomers, polymers, dendrimers, ionomers, copolymers such as for example, block copolymers, random copolymers, graft copolymers, star block copolymers; or the like, or a combination including at least one of the foregoing polymers can be used. Thermoplastic polymers or thermosetting polymers can be used. Examples of suitable thermoplastic polymers include polyacrylates, polymethacrylates, polyamides, polyesters, polyolefins, polycarbonates, polystyrenes, polyesters, polyamideimides, polyarylates, polyarylsulfones, polyethersulfones, polyphenylene sulfides, polysulfones, polyimides, polyetherimides, polyetherketones, polyether etherketones, polyether ketone ketones, polysiloxanes, polyurethanes, polyarylene ethers, polyethers, polyether amides, polyether esters, or the like, or a combination including at least one of the foregoing thermoplastic polymers. Some more possible examples of suitable thermoplastic polymers include, but are not limited to, amorphous and semi-crystalline thermoplastic polymers and polymer blends, such as: polyvinyl chloride, linear and cyclic polyolefins, chlorinated polyethylene, polypropylene, and the like; hydrogenated polysulfones, ABS resins, hydrogenated polystyrenes, syndiotactic and atactic polystyrenes, polycyclohexyl ethylene, styrene-acrylonitrile copolymer, styrene-maleic anhydride copolymer, and the like; polybutadiene, polymethylmethacrylate (PMMA), methyl methacrylate-polyimide copolymers; polyacrylonitrile, polyacetals, polyphenylene ethers, including, but not limited to, those derived from 2,6-dimethylphenol and copolymers with 2,3,6-trimethylphenol, and the like; ethylene-vinyl acetate copolymers, polyvinyl acetate, ethylene-tetrafluoroethylene copolymer, aromatic polyesters, polyvinyl fluoride, polyvinylidene fluoride, and polyvinylidene chloride.

In some embodiments, the thermoplastic polymer used in the methods disclosed herein as a substrate is made of a polycarbonate. The polycarbonate may be an aromatic polycarbonate, an aliphatic polycarbonate, or a polycarbonate including both aromatic and aliphatic structural units.

As used herein, the term “polycarbonate” includes compositions having structural units of the formula XV:

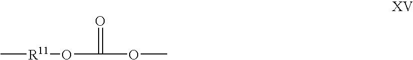

wherein R¹¹ is an aliphatic, aromatic or a cycloaliphatic radical. In an embodiment, the polycarbonate includes structural units of the formula XVI:

-A¹-Y¹-A²- XVI

wherein each of A¹ and A² is a monocyclic divalent aryl radical and yl is a bridging radical having zero, one, or two atoms which separate A¹ from A². In an exemplary embodiment, one atom separates A¹ from A². Non-limiting examples of radicals include —O—, —S—, —S(O)—, —S(O)₂—, —C(O)—, methylene, cyclohexyl-methylene, 2-ethylidene, isopropylidene, neopentylidene, cyclohexylidene, cyclopentadecylidene, cyclododecylidene, and adamantylidene. Some examples of such bisphenol compounds are bis(hydroxyaryl)ethers such as 4,4′-dihydroxy diphenylether, 4,4′-dihydroxy-3,3′-dimethylphenyl ether, or the like; bis(hydroxy diaryl)sulfides, such as 4,4′-dihydroxy diphenyl sulfide, 4,4′-dihydroxy-3,3′-dimethyl diphenyl sulfide, or the like; bis(hydroxy diaryl)sulfoxides, such as, 4,4′-dihydroxy diphenyl sulfoxides, 4,4′-dihydroxy-3,3′-dimethyl diphenyl sulfoxides, or the like; bis(hydroxy diaryl)sulfones, such as 4,4′-dihydroxy diphenyl sulfone, 4,4′-dihydroxy-3,3′-dimethyl diphenyl sulfone, or the like; or combinations including at least one of the foregoing bisphenol compounds. In one embodiment, zero atoms separate A¹ from A², with an illustrative example being biphenol. The bridging radical Y¹ can be a hydrocarbon group, such as, for example, methylene, cyclohexylidene or isopropylidene, or aryl bridging groups.

Any of the dihydroxy aromatic compounds known in the art can be used to make the polycarbonates. Examples of dihydroxy aromatic compounds include, for example, compounds having formula XVII

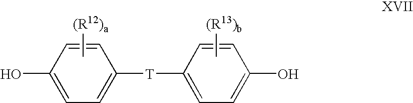

wherein R¹⁶ and R¹⁷ each independently represent a halogen atom, or a aliphatic, aromatic, or a cycloaliphatic radical; a and b are each independently integers from 0 a to 4; and T represents one of the groups having formula XVIII

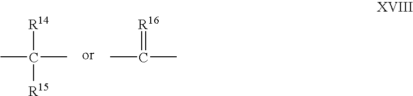

wherein R¹⁴ and R¹⁵ each independently represent a hydrogen atom or a aliphatic, aromatic or a cycloaliphatic radical; and R¹⁶ is a divalent hydrocarbon group. Some illustrative, non-limiting examples of suitable dihydroxy aromatic compounds include dihydric phenols and the dihydroxy-substituted aromatic hydrocarbons such as those disclosed by name or structure (generic or specific) in U.S. Pat. No. 4,217,438. Polycarbonates including structural units derived from bisphenol A may be selected since they are relatively inexpensive and commercially readily available. A nonexclusive list of specific examples of the types of bisphenol compounds that may be represented by structure (XVII) includes the following: 1,1-bis(4-hydroxyphenyl)methane; 1,1-bis(4-hydroxyphenyl)ethane; 2,2-bis(4-hydroxyphenyl)propane (hereinafter “bisphenol A” or “BPA”); 2,2-bis(4-hydroxyphenyl)butane; 2,2-bis(4-hydroxyphenyl)octane; 1,1-bis(4-hydroxyphenyl)propane; 1,1-bis(4-hydroxyphenyl)n-butane; bis(4-hydroxyphenyl)phenylmethane; 2,2-bis(4-hydroxy-3-methylphenyl)propane (hereinafter “DMBPA”); 1,1-bis(4-hydroxy-t-butylphenyl)propane; bis(hydroxyaryl)alkanes such as 2,2-bis(4-hydroxy-3-bromophenyl)propane; 1,1-bis(4-hydroxyphenyl)cyclopentane; 9,9′-bis(4-hydroxyphenyl)fluorene; 9,9′-bis(4-hydroxy-3-methylphenyl)fluorene; 4,4′-biphenol; and bis(hydroxyaryl)cycloalkanes such as 1,1-bis(4-hydroxyphenyl)cyclohexane and 1,1-bis(4-hydroxy-3-methylphenyl)cyclohexane (hereinafter “DMBPC”); and the like, as well as combinations including at least one of the foregoing bisphenol compound.

Polycarbonates can be produced by any of the methods known in the art. Branched polycarbonates are also useful, as well as blends of linear polycarbonates and branched polycarbonates. In one embodiment, the polycarbonates may be based on bisphenol A. In one embodiment, the weight average molecular weight of the polycarbonate is about 5,000 to about 100,000 atomic mass units. In one embodiment, the weight average molecular weight of the polycarbonate is about 5000 to about 10000 atomic mass units, about 10000 to 20000 atomic mass units, about 20000 to 40000 atomic mass units, about 40000 to 60000 atomic mass units, about 60000 to 80000 atomic mass units, or about 80000 to 100000 atomic mass units. Other specific examples of a suitable thermoplastic polymer for use in forming the holographic data storage media include Lexan®, a polycarbonate; and Ultem®, an amorphous polyetherimide, both of which are commercially available from SABIC IP.

Examples of useful thermosetting polymers include those selected from the group consisting of an epoxy, a phenolic, a polysiloxane, a polyester, a polyurethane, a polyamide, a polyacrylate, a polymethacrylate, and a combination including at least one of the foregoing thermosetting polymers.

In one embodiment, a holographic recording medium is provided that includes an optically transparent substrate. The optically transparent substrate includes a photochemically active dye, a protonated form of the photochemically active dye, and a photo-product of the photochemically active dye. The protonated form of the photochemically active dye is a composition having a structure as shown in formula I, and the photochemically active dye is a composition having a structure as shown in formula II. The photo-product is patterned within the optically transparent substrate to provide an optically readable datum contained within a volume of the holographic recording medium. In one embodiment, the optically readable datum comprises a volume element having an average refractive index that differs from a corresponding volume element of the optically transparent substrate, said volume element being characterized by a change in the average refractive index relative to the refractive index of the corresponding volume element prior to the at least one photo-product being patterned.

In one embodiment, a method uses the holographic recording medium. The method includes irradiating an optically transparent substrate. The substrate includes a photochemically active dye with an incident light at a wavelength in a range of from about 300 nanometers to about 1000 nanometers. The irradiation forms an optically readable datum and a photo-product of the photochemically active dye. The holographic recording medium is exposed to an acid, and at least part of the photochemically active dye is protonated. The protonated form of the photochemically active dye is a composition having a structure as shown in formula I, and the photochemically active dye is a composition having a structure as shown in formula II.

In one embodiment, an optical writing and reading method includes patterning a holographic recording medium with a signal beam possessing data and a reference beam simultaneously to create a hologram. This patterning partly converts the photochemically active dye into a photo-product. The holographic recording medium is exposed to an acid, resulting in at least part of the photochemically active dye forming a protonated form of the photochemically active dye. Information in the signal beam can be stored as a hologram in the holographic recording medium. The holographic recording medium is contacted with a read beam to read the data contained in the hologram-diffracted light.

The holographic recording medium includes an optically transparent substrate. The optically transparent substrate includes a photochemically active dye. The protonated form of the photochemically active dye is a composition having a structure as shown in formula I, and the photochemically active dye is a composition having a structure as shown in formula II. In one embodiment, the read beam has a wavelength that is shifted by an amount in a range of about 0.001 nanometers to about 500 nanometers relative to the signal beam's wavelength. In another embodiment, the read beam wavelength is not shifted relative to the signal beam's wavelength.

In one embodiment, a method includes patterning a holographic recording medium in a holographic recording medium article with an electromagnetic radiation having a first wavelength, forming a modified optically transparent substrate comprising at least one photo-product of the at least one photochemically active dye, and at least one optically readable datum stored as a hologram, exposing the modified optically transparent substrate to acid; resulting in at least part of the photochemically active dye forming a protonated form of the photochemically active dye, and contacting the holographic recording medium in the article with electromagnetic energy having a second wavelength to read the hologram. The holographic recording medium includes an optically transparent substrate. The optically transparent substrate includes a photochemically active dye. The photochemically active dye is a composition having a structure as shown in formula II and the protonated form of the photochemically active dye is a composition having a structure as shown in formula I.

In one embodiment, the second wavelength is shifted by an amount in a range of from about 0.001 nanometers to about 500 nanometers relative to the first wavelength. In one embodiment, the first wavelength is not the same as the second wavelength. In one embodiment, the first wavelength is the same as the second wavelength. In another embodiment, the read beam wavelength is not shifted relative to the signal beam's wavelength.

In various embodiments, the photochemically active dye may be selected and utilized on the basis of several characteristics, including the ability to change the refractive index of the dye upon exposure to light; the efficiency with which the light creates the refractive index change; and the separation between the wavelength at which the dye shows a maximum absorption and the desired wavelength or wavelengths to be used for storing and/or reading the data. The choice of the photochemically active dye depends upon many factors, such as sensitivity (S) of the holographic recording media, concentration (N₀) of the photochemically active dye, the dye's absorption cross section (σ) at the recording wavelength, the quantum efficiency (QE) of the photochemical conversion of the dye, and the refractive index change per unit dye density (i.e., Δn/N₀). Of these factors, QE, Δn/N₀, and σ are more important factors which affect the sensitivity (S) and also information storage capacity (M/#). In one embodiment, photochemically active dyes that show a high refractive index change per unit dye density (Δn/N₀), a high quantum efficiency in the photochemical conversion step, and a low absorption cross-section at the wavelength of the electromagnetic radiation used for the photochemical conversion are selected.

In one embodiment, the photochemically active dye may be one that is capable of being written and read by electromagnetic radiation. In one embodiment, it may be desirable to use dyes that can be written (with a signal beam) and read (with a read beam) using actinic radiation i.e., radiation having a wavelength from about 300 nanometers to about 1000 nanometers. The wavelengths at which writing and reading may be accomplished may be in a range of from about 300 nanometers to about 800 nanometers. In one embodiment, the writing and reading are accomplished at a wavelength of about 400 nanometers to about 500 nanometers, at a wavelength of about 500 nanometers to about 550 nanometers, or at a wavelength of about 550 nanometers to about 600 nanometers. In one embodiment, the reading wavelength is shifted by a minimum amount of nanometers up to about 400 nanometers relative to the writing wavelength. Exemplary wavelengths at which writing and reading are accomplished are about 405 nanometers and about 532 nanometers.

In one embodiment, the photochemically active dye may be admixed with other additives to form a photo-active material. Examples of such additives include heat stabilizers, antioxidants, light stabilizers, plasticizers, antistatic agents, mold releasing agents, additional resins, binders, blowing agents, and the like, as well as combinations of the foregoing additives. In one embodiment, the photo-active materials may be used for manufacturing holographic recording media.

In one embodiment, a holographic recording medium is manufactured. The method of manufacturing includes the steps of forming a film, an extrudate, or an injection molded part of an optically transparent substrate including a photochemically active dye, the optically transparent substrate comprises the optically transparent plastic material and the photochemically active dye, exposing the film, the extrudate, or the injection molded part to an acid, and resulting in at least part of the photochemically active dye forming a protonated form of the photochemically active dye. The photochemically active dye is a composition having a structure as shown in formula II and the protonated form of the photochemically active dye is a composition having a structure as shown in formula I. The film formation may include thermoplastic extrusion. The film formation may include solvent casting. The film formation may include thermoplastic molding.

In one embodiment, a method for rendering a permanent hologram in a holographic recording medium is provided. The method includes irradiating an optically transparent substrate comprising a photochemically active dye with an incident light at a wavelength in a range of from about 300 nanometers to about 1000 nanometers, patterning a holographic recording medium with a signal beam possessing data and a reference beam simultaneously to create a hologram, and thereby partly converting the photochemically active dye into a photo-product, resulting in forming the holographic recording medium comprising an optically readable datum and a photo-product of the photochemically active dye, and exposing the holographic recording medium to an acid, resulting in the conversion of the photochemically active dye to a protonated form of the photochemically active dye. The photochemically active dye is a composition having a structure as shown in formula II and the protonated form of the photochemically active dye is a composition having a structure as shown in formula I.

In one embodiment, a holographic recording medium is provided. The holographic recording medium includes an optically transparent substrate. The optically transparent substrate includes a photochemically active dye, a protonated form of the photochemically active dye, a photo-product of the photochemically active dye and a protonated form of the photo-product of the photochemically active dye. The protonated form of the photochemically active dye is a composition having a structure as shown in formula I, and the photochemically active dye is a composition having a structure as shown in formula II. The photo-product is patterned within the optically transparent substrate to provide an optically readable datum contained within a volume of the holographic recording medium.

### EXAMPLES

The following examples illustrate methods and embodiments in accordance with the invention, and as such should not be construed as imposing limitations upon the claims. Unless specified otherwise, all components are commercially available from common chemical suppliers such as Alpha Aesar, Inc. (Ward Hill, Mass.), Spectrum Chemical Mfg. Corp. (Gardena, Calif.), and the like.

### Example 1

### Preparation of a Dye

Step A: Preparation of Phenylhydroxylamine.

Ammonium chloride (20.71 grams, 0.39 moles), de-ionized water (380 milliliters), nitrobenzene (41.81 grams, 0.34 moles), and ethanol (420 milliliters, 95 percent) are added to a 1-liter, 3-neck round-bottom flask equipped with a mechanical stirrer, thermometer, and nitrogen inlet. The resultant reaction mixture is cooled to 15 degrees Celsius using an ice water bath. Zinc powder (46.84 grams, 0.72 moles) is added to the cooled mixture in portions, and over a period of about 0.5 hours while ensuring that the temperature does not exceed 25 degrees Celsius. After the complete addition of the zinc, the reaction mixture is warmed to room temperature. The warmed mixture is stirred for half an hour and is then filtered to remove zinc salt and unreacted zinc. The filter cake (i.e., the zinc salt) is first washed with hot water (about 200 milliliters) and then is washed with methylene chloride (about 100 milliliters). The filtrate is extracted with methylene chloride (about 100 milliliters). The methylene chloride layers (obtained from the filter cake wash and filtrate extract) are combined, washed with brine (about 100 milliliters), dried over sodium sulfate, and the methylene chloride is evaporated. The product is dried in a vacuum oven for about 24 hours to give 17.82 grams of phenylhydroxylamine as a fluffy light yellow solid.

Step B: Preparation of alpha-(4-dimethylamino)styryl-N-phenyl nitrone.

To a 1 liter, 3-neck round-bottom flask equipped with a mechanical stirrer and a nitrogen inlet is added phenylhydroxylamine (27.28 grams, 0.25 moles), 4-dimethylaminocinnamaldehyde (43.81 grams, 0.25 moles) and ethanol (250 milliliters) resulting in a bright orange colored mixture. To the resultant mixture, methanesulfonic acid (250 microliters) is added using a syringe. The resultant mixture turns to a deep red color solution with the dissolution of all the solids. Within about five minutes an orange solid is formed. Pentane (˜300 ml) is added to the mixture to facilitate stirring. The solid is filtered and dried in a vacuum oven at 80 degrees Celsius for about 24 hours to give 55.91 grams of alpha-(4-dimethylamino)styryl-N-phenylnitrone as a bright orange solid.

### Example 2

### Preparation of Dye

Step A: Preparation of 4-carbethoxyphenyl hydroxylamine

Ammonium chloride (9.2 grams, 0.17 moles), de-ionized water (140 milliliters), p-nitroethylbenzoate (29.28 grams, 0.15 moles), and ethanol (150 milliliters, 95 percent) are added to a 500 milliliter, 3-neck round-bottom flask equipped with a mechanical stirrer, thermometer, and nitrogen inlet. The resultant reaction mixture is cooled to 15 degrees Celsius using an ice water bath. Zinc powder (21.82 grams, 0.34 moles) is added to the cooled mixture in portions, and over a period of about 0.25 hours while ensuring that the temperature does not exceed 15 degrees Celsius. After the complete addition of the zinc, the reaction mixture is warmed to room temperature. The warmed mixture is stirred for one hour and is then filtered to remove zinc salt and unreacted zinc. The filter cake (i.e., the zinc salt) is first washed with hot water (about 200 milliliters) and then is washed with methylene chloride (about 100 milliliters). The filtrate is extracted with methylene chloride (about 100 milliliters). The methylene chloride layers (obtained from the filter cake wash and filtrate extract) are combined, washed with brine (about 100 milliliters), dried over sodium sulfate, and the methylene chloride is evaporated. The product is dried in a vacuum oven for about 24 hours to give 20.04 grams of 4-carbethoxyphenyl hydroxylamine as a fluffy light yellow solid.

Step B: Preparation of alpha-(4-dimethylamino)styryl-N-4-carbethoxyphenylnitrone.

To a 100 milliliters 3-neck round-bottom flask equipped with a mechanical stirrer and a nitrogen inlet is added 4-carbethoxyphenyl hydroxylamine (4.53 grams, 0.025 moles), 4-dimethylamino cinnnamaldehyde (4.38 grams, 0.025 moles) and ethanol (25 milliliters) resulting in a bright orange colored mixture. To the resultant mixture methanesulfonic acid (2 microliters) is added using a syringe. The resultant mixture turns to a deep red color solution with the dissolution of all solids. Within about five minutes a red solid is formed. The solid is filtered, washed with pentane (100 milliliters) and dried in a vacuum oven at 50 degrees Celsius for about 24 hours to give 6.23 grams of alpha-(4-dimethylamino)styryl-N-4-carbethoxyphenyl nitrone.

### Example 3

### Procedure for Preparing Solution Samples

About 2 milligrams of the dye prepared in Example 1 or in Example 2 are added to acetonitrile (100 milliliters). The resultant mixture is stirred for about 2 hours or until complete dissolution of the dye in the acetonitrile.

### Example 4

### Sample Evaluation—Solution Samples

Procedure for measuring UV-Visible spectra of the photochemically active dyes. All spectra are recorded on a Cary/Varian 300 UV-Visible spectrophotometer using solutions. Spectra are recorded in the range of about 300 nanometers to a about 800 nanometers. Solution samples prepared in Example 3 using the dye prepared in Example 2 are taken in 1 centimeter quartz cuvettes and acetonitrile is taken as the blank solvent to be placed in the reference beam path for the UV-Visible measurement. Concentrated hydrochloric acid is added to the cuvettes containing the solution samples with a microliter pipette. The UV-Visible spectra for each of the samples is measured before and after the addition of the concentrated hydrochloric acid to the cuvettes.

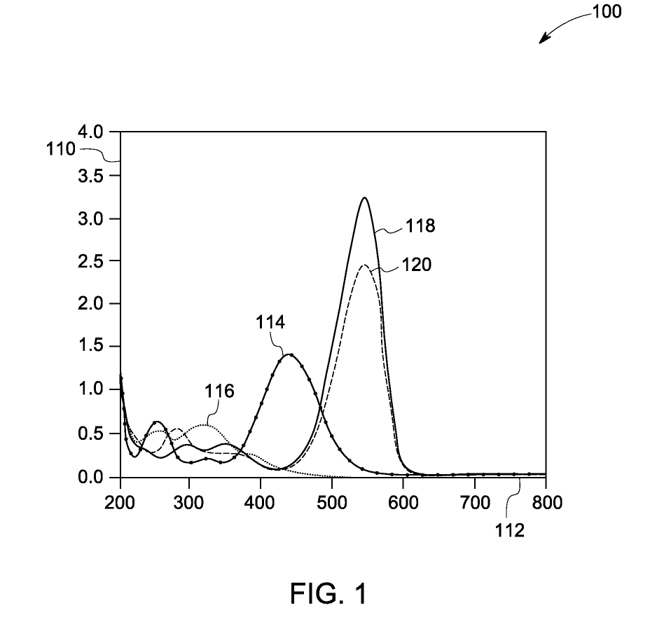

With reference to FIG. 1, a graph 100 shows a change in absorbance of a photochemically active dye according to an embodiment of the invention. The graph has absorbance 110 versus wavelength of light in nanometers 112. Curve 114 is absorbance for the dye in the visible region before photobleaching i.e., before exposure to UV and before the addition of concentrated hydrochloric acid. Curve 114 has an absorption maxima at about 441 nanometers. Curve 116 is the absorbance of the UV exposed form of the dye before the addition of concentrated hydrochloric acid having an absorption maxima at about 312 nanometers. Curve 118 is the absorbance for the dye before photo-bleaching and after the addition of concentrated hydrochloric acid having an absorption maxima at about 548 nanometers. Curve 120 is absorbance of the UV exposed form of the dye after the addition of concentrated hydrochloric acid having an absorption maxima at about 548 nanometers. The graph indicates that the dye is photosensitive to 532 nanometers and 405 nanometers laser light and rapidly photobleaches upon exposure to UV, resulting in a decrease in the absorption maxima from about 441 nanometers to about 312 nanometers. However, if the dye is protonated with an acid there is an increase in the absorption maxima in the UV-Visible region from about 441 nanometers to about 548 nanometers. Also, when the protonated dye is exposed to UV there is not much change in the absorption maxima indicating the decreased photosensitivity of the dye in the protonated form.

### Example 5

### Procedure for Preparing Spin Coated Samples

Spin coated samples are prepared by dissolving 32 milligrams of dye prepared in Example 2 and 1 grams PMMA in 10 milliliters of tetrachloroethane. This solution is poured onto a glass slide and spin-coated at 1000 rpm, followed by drying on a hotplate maintained at 45 degrees Celsius for about 30 minutes. The samples are dried in a vacuum oven at 40 degrees Celsius for about 12 hours. The sample contains about 3.2 weight percent of dye prepared in Example 2 in PMMA, spin-coated to a thickness of about 500 nanometers. Photobleaching of the sample is conducted with a handheld broadband UV-Visible light source with about 365 nanometers/30 milliWatts peak output. The film samples are exposed to hydrochloric acid vapor for about 2 minutes from an aqueous concentrated hydrochloric acid solution.

### Example 6

### Sample Evaluation of Spin Coated Samples

Procedure for measuring UV-Visible spectra of the spin coated samples. All spectra recorded using time resolved UV-Visible spectra are obtained on an Ocean Optics fiber coupled USB2000 spectrometer under simultaneous laser irradiation at about 532 nanometers. Absorption spectra are recorded in the range of about 200 nanometers to about 800 nanometers. Samples are protonated by placing the samples at the mouth of a bottle containing aqueous concentrated hydrochloric acid for about 2 minutes to about 30 minutes depending on the sample thickness. Acid vapors diffuse through the sample, thus protonating the dye in the sample. Samples are prepared by spin-coating thin films having a thickness of about 500 nanometers, onto silicon wafers with different levels of dye loading i.e., 0.45, 1.06, 1.64, 3.22 and 4.97. The samples are measured over a wavelength range of from about 200 nanometers to about 800 nanometers and at multiple angles and the analysis is typically done with a general oscillator model. Refractive index is obtained using Kramer-Kronig relationship by fitting the modeled absorption to the measured absorption. The films are measured in their initial state i.e., before protonation and after protonation.

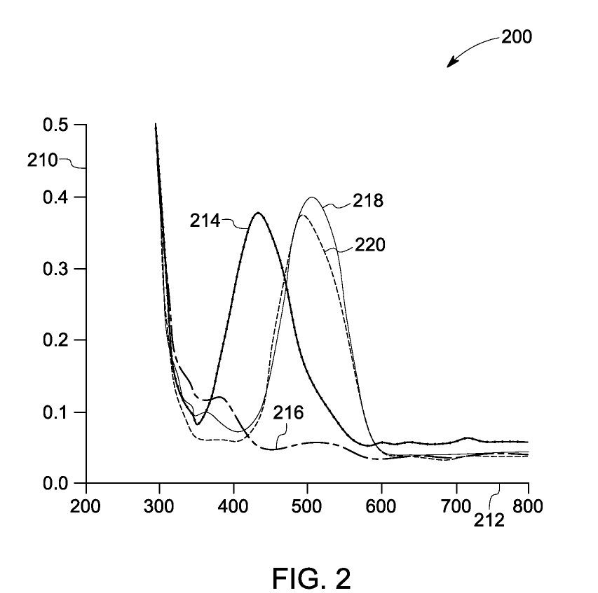

With reference to FIG. 2, a graph 200 shows a change in absorbance of a photochemically active dye according to an embodiment of the invention. The graph has absorbance 210 versus wavelength of light in nanometers 212. Curve 214 is absorbance for the dye in the visible region before photobleaching and before the addition of concentrated hydrochloric acid. Curve 214 has an absorption maxima at about 435 nanometers. Curve 216 is the absorbance of the UV exposed form of the dye before the addition of concentrated hydrochloric acid having an absorption maxima at about 390 nanometers. Curve 218 is the absorbance for the dye before photo-bleaching and after the addition of concentrated hydrochloric acid having an absorption maxima at about 500 nanometers. Curve 220 is absorbance of the UV exposed form of the dye after the addition of concentrated hydrochloric acid having an absorption maxima at about 500 nanometers. The graph indicates a similar behavior of the dye in the spin coated sample as shown above in the solution samples. The graph indicates that the dye is photosensitive to 532 nanometers and 405 nanometers laser light and photobleaches upon exposure to UV, resulting in a decrease in the absorption maxima from about 435 nanometers to about 390 nanometers. However, if the dye is protonated with an acid there is an increase in the absorption maxima in the UV-Vis region from about 435 nanometers to about 500 nanometers. Also, when the protonated dye is exposed to UV there is not much change in the absorption maxima indicating the decreased photosensitivity of the dye in the protonated form.

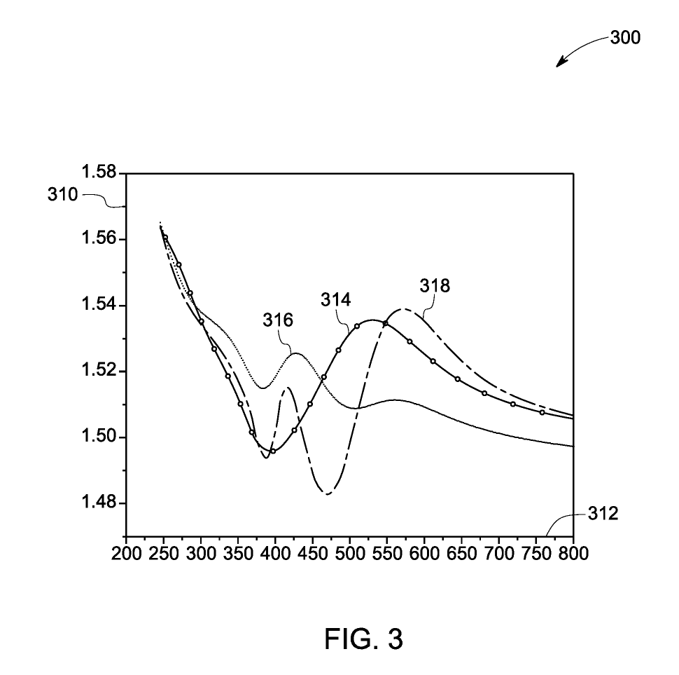

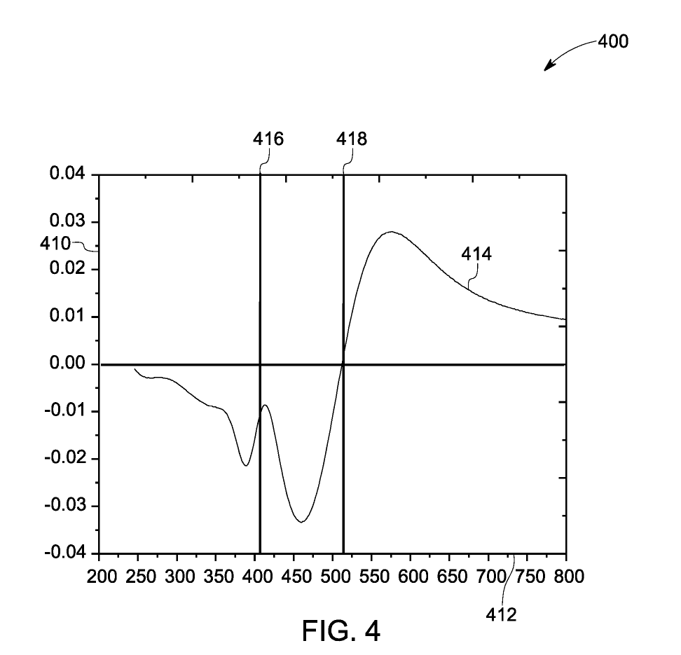

The absorption reported in the tables is calculated by subtracting the average baseline in the range of 700 to 800 nanometers for each sample tested from the measured absorption at either 405 nanometers or 532 nanometers. Since these compounds do not absorb in the 700 to 800 nanometers range, this correction removes the apparent absorption caused by reflections off the surfaces of the disc and provides a more accurate representation of the absorbance of the dye. The polymers used in these examples have little or no absorption at 405 nanometers or 532 nanometers. The results of these measurements are shown in FIG. 3, FIG. 4, and Table 1.

With reference to FIG. 3, a graph 300 shows a change in refractive index of a photochemically active dye according to an embodiment of the invention. The graph has refractive index 310 versus wavelength of light in nanometers 312. Curve 314 is refractive index for the dye in the visible region before photobleaching and before the addition of concentrated hydrochloric acid having a maximum refractive index of about 1.535. Curve 316 is the refractive index of the UV exposed form of the dye before the addition of concentrated hydrochloric acid having a maximum refractive index of about 1.525. Curve 318 is the refractive index for the dye before photo-bleaching and after the addition of concentrated hydrochloric acid having a maximum refractive index at about 1.539.

With reference to FIG. 4, a graph 400 shows a refractive index change of a photosensitive material according to an embodiment of the invention. The graph has difference in refractive index (Δ RI) 410 versus wavelength of light in nanometers 412. Curve 414 shows a refractive index change for the spin coated sample prepared in Example 5. An activation region of light of a determined wavelength has a lower bound 416 at about 405 nanometers, and an upper bound 418 at about 532 nanometers. The upper and lower bounds define an area which includes the RI difference between the protonated form of the dye and the bleached form of the dye obtained when the dye in its protonated and non-protonated form absorbs light and affects the conformational change to affect the refractive index of the host article. The change in refractive index of the spin coated sample prepared in Example 5 measured at 405 nanometers and at 532 nanometers is included in Table 1 below. Table 1 includes the maximum Δn between an unbleached and a bleached sample and between a protonated and a bleached sample.

TABLE 1

| Spin Coated Sample | At 405 | At 532 | |
|---|---|---|---|
| of Example 5 | nanometers | nanometers | Maximum Δn |
| Δn between unbleached and | −0.0036 | 0.014 | −0.025 at 415 |
| bleached | | | nanometers |
| Δn between protonated and | −0.011 | 0.0158 | −0.035 at 460 |
| bleached | | | nanometers |

As discussed above, the dye prepared in Example 2, would ideally be exposed at 532 nanometers to write a hologram, which is followed by exposure to acid vapors for 2 minutes, enhancing the refractive index and simultaneously rendering the dye photoinsensitive. The recommended read-out wavelength is 450 nanometers for spectroscopic ellipsometry. The dye is photosensitive to 532 nanometers and 405 nanometers laser light and rapidly photobleaches upon exposure to UV. However, if the dye is protonated with an acid, the photosensitivity is dramatically reduced and a strong shift of the absorption band to a longer wavelength is observed.

### Example 7

### Preparation of Dye—Polymer Mixture

Ten kilograms of pelletized polystyrene PS1301 (obtained from Nova Chemicals) is ground to a coarse powder in a Retsch mill and dried in a circulating oven maintained at 80 degrees Celsius for 12 hours. In a 10 liter Henschel mixer, 6.5 kilograms of the dry polystyrene powder and 195 grams of alpha-(4-dimethylamino)styryl-N-phenylnitrone are blended to form a homogeneous orange powder. The powder is fed into a Prism (16 mm) twin-screw extruder at 185 degrees Celsius to give 6.2 kilograms of dark orange colored pellets with a dye content of about 3 weight percent. The conditions used for extruding are included in Table 2.

TABLE 2

| Extrusion Parameters | Values |
|---|---|
| Screw (revolutions per minute) | 300 |
| Feeder Rate (units) | 4.8-6.3 |
| | (at 50 percent) |
| Torque (percent) | 68-72 |
| Temp Zone 1 (degrees Celsius) | 160-200 |
| Temp Zones 2-9 (degrees Celsius) | 170-190 |

### Example 8

### Preparation of Dye—Polymer Mixture

The extruded pellets obtained in Example 7 are dried in vacuum oven at temperatures of nearly 40 degrees Celsius below the glass transition temperature of the polymer. Optical quality discs are prepared by injection molding blends (prepared as described above) with a Sumitomo, SD-40E all-electrical commercial CD/DVD (compact disc/digital video disc) molding machine (available from Sumitomo Inc.). The molded discs have a thickness in a range from about 500 micrometers to about 1200 micrometers. Mirrored stampers are used for both surfaces. Cycle times are generally set to about 10 seconds. Molding conditions are varied depending upon the glass transition temperature and melt viscosity of the polymer used, as well as the photochemically active dye's thermal stability. Thus the maximum barrel temperature is controlled to be in a range of from about 200 degrees Celsius to about 375 degrees Celsius. The molded discs are collected and stored in the dark.

### Example 9

### Procedure for Preparing Molded Disc

Conditions used for molding OQ (Optical Grade) polystyrene based blends of the photochemically active dyes are shown in Table 3.

TABLE 3

| | Polystyrene |
|---|---|
| Molding Parameters | Blend |
| Barrel Temperature (Rear) (degrees Celsius) | 205 |
| Barrel Temperature (Front) (degrees Celsius) | 200 |
| Barrel Temperatuer (Nozzle) (degrees Celsius) | 200 |
| Melt Temperature (degrees Celsius) | 200-250 |
| Mold Temperature (degrees Celsius) | 50-70 |
| Total cycle Time (sec) | 3-12 |
| Switch Point (inch) | 0.7 |
| Injection Transition (inch) | 0.2 |
| Injection Boost Pressure (psi) | 1100 |
| Injection Hold pressure (psi) | 400 |
| Injection Velocity (millimiter per second) | 60-150 |

### Example 10

### Method of Use

Procedure for Recording of the Hologram

For recording of the hologram at either 532 nanometers or 405 nanometers, both the reference beam and the signal beam are incident on the test sample at oblique angles of 45 degrees. The sample is positioned on a rotary stage, which is controlled by a computer. Both the reference and signal beams have the same optical power and are polarized in the same direction (parallel to the sample surface). The beam diameters (1/e²) are 4 millimeters. A color filter and a small pinhole are placed in front of the detector to reduce optical noise from the background light. A fast mechanical shutter in front of the laser controls the hologram recording time. In the 532 nanometers setup, a red 632 nanometers beam is used to monitor the dynamics during hologram recording. The recording power for each beam varies from 1 milliWatt to 100 milliWatts and the recording time varies from 10 milliseconds to about 5 seconds. The diffracted power from a recorded hologram is measured from a Bragg detuning curve by rotating the sample disc by 0.2 to 0.4 degrees. The reported values are corrected for reflections off the sample surface. The power used to readout the holograms is two to three orders of magnitude lower than the recording power in order to minimize hologram erasure during readout. Results of the UV-Visible absorption spectra measurements and the diffraction efficiencies of the dye prepared in Example 1 that are used for preparing the discs in Example 9 is included in Table 4 below.

### Example 11

### Sample Evaluation

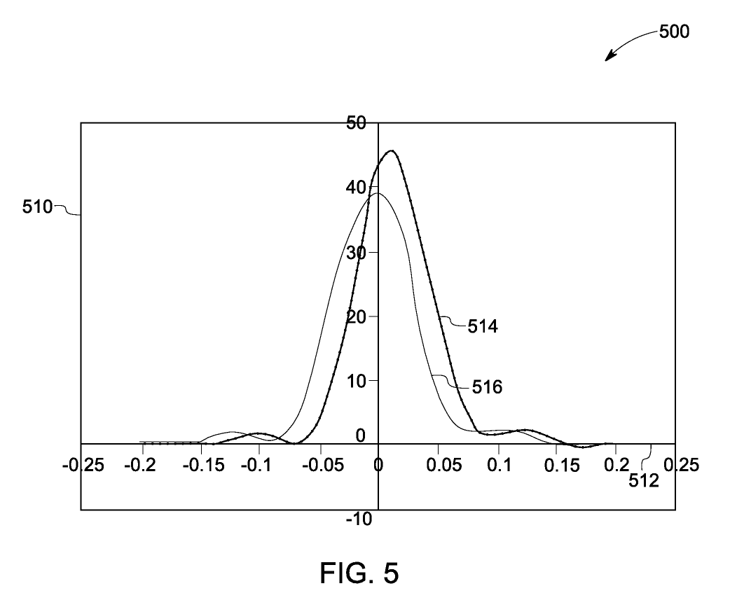

Samples prepared in Example 9 are protonated by placing the samples at the mouth of the bottle containing aqueous HCl for about 2 minutes to about 30 minutes depending on sample thickness/configuration. Acid vapors diffuse through the sample, thus protonating it. The diffractions efficiencies of the samples prepared in Example 9 are measured in their initial state i.e., before protonation and after protonation. It is observed that upon exposure to acid, there is a strong shift of the absorption band to longer wavelength enhancing the refractive index and thus, increasing the diffraction efficiency. Also, exposure to acid dramatically reduces the photosensitivity, thus enhancing the hologram stability. Diffraction efficiency measurements for molded disc (containing 3 weight percent dye prepared in Example 1) before and after protonation are shown in Table 4 and FIG. 5.

TABLE 4
Diffraction efficiency measurements for molded disc

Diffraction efficiency (corrected)

| | before | |
|---|---|---|
| | protonation | after protonation |
| 3 weight percent dye | 39.2 | 45.8 |
| in polystyrene | | |

Thickess of disc = 600 microns

With reference to FIG. 5, a graph 500 shows a diffraction efficiency change of a photosensitive material according to an embodiment of the invention. The graph has diffraction efficiency 510 versus angle of diffraction in degrees 512. Curve 514 is absorbance for the molded disc prepared in Example 9 before protonation and Curve 516 is absorbance for the molded disc prepared in Example 9 after protonation. There is a marked increase in the diffraction efficiency after protonation when compared to that before protonation.

### Example 12

### Procedure for Preparing Solvent Cast Samples

1 gram of polystyrene pellets are dissolved in 10 milliliters of methylene chloride and stirred for about 2 hours or till the polystyrene pellets are completely dissolved in the methylene chloride. (4-dimethylamino)styryl-N-phenyl nitrone (50 milligrams) is added to the polymer solution and stirred for about 2 hours or till the nitrone is completely dissolved in the methylene chloride. Solvent cast samples are made by pouring the dye-polystyrene solution inside a metal ring (5 centimeter radius) resting over a glass substrate. The assembly of the metal ring placed over the glass substrate is placed over a hot plate maintained at a temperature of about 40 degrees Celsius. The assembly is covered with an inverted funnel to allow slow evaporation of methylene chloride. Dried dye-doped polystyrene films are recovered after about 4 hours. The dye-doped polystyrene films contain 5 weight percent of the dye.

### Example 13

### Method of Rendering the Hologram Permanent

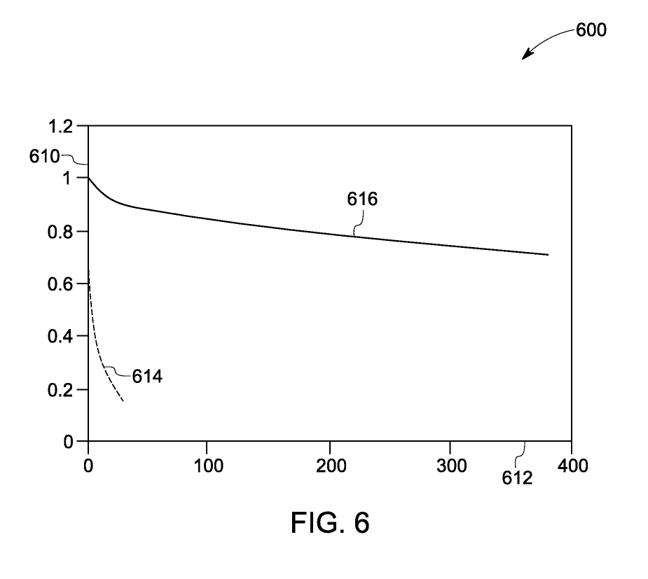

The films are subjected to a hologram erasure beam 532 nanometers/100 milliwatts for about 30 to about 400 seconds before protonation and after protonation. Table 5 indicates the decrease in the diffraction efficiency of the protonated sample is lower than the decrease in the diffraction efficiency of the sample before protonation. The effect of the hologram erasure beam on a sample before protonation and on a sample after protonation is provided in FIG. 6.

TABLE 5

Diffraction Efficiency (Normalized)

| Sample prepared | Before | |
|---|---|---|
| in Example 12 | Protonation | After Protonation |
| DE Before exposure | 100 | 100 |
| DE After 30 s exposure | 16 | 89 |

With Reference to FIG. 6, a graph 600 shows a hologram erasure measurement of an article according to an embodiment of the invention. The graph has diffraction efficiency 610 versus hologram erasure time in seconds 612. Curve 614 is change in diffraction efficiency with time when subjected to the hologram erasure beam observed in a sample before protonation. Curve 614 is change in diffraction efficiency with time when subjected to the hologram erasure beam observed in a sample after protonation. The amount of time taken to erase the hologram in a sample before protonation is about 30 seconds and time taken to erase the hologram in a sample after protonation is about 380 seconds. This indicates that protonation renders the dye insensitive to the bleaching wavelength thus rendering the hologram permanent.

The singular forms “a”, “an”, and “the” include plural referents unless the context clearly dictates otherwise. “Optional” or “optionally” means that the subsequently described event or circumstance may or may not occur, and that the description includes instances where the event occurs and instances where it does not. Approximating language, as used herein throughout the specification and claims, may be applied to modify any quantitative representation that could permissibly vary without resulting in a change in the basic function to which it is related. Accordingly, a value modified by a term or terms, such as “about” and “substantially”, are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise. Molecular weight ranges disclosed herein refer to molecular weight as determined by gel permeation chromatography using polystyrene standards.

While the invention has been described in detail in connection with a number of embodiments, the invention is not limited to such disclosed embodiments. Rather, the invention can be modified to incorporate any number of variations, alterations, substitutions or equivalent arrangements not heretofore described, but which are commensurate with the scope of the invention. Additionally, while various embodiments of the invention have been described, it is to be understood that aspects of the invention may include only some of the described embodiments. Accordingly, the invention is not to be seen as limited by the foregoing description, but is only limited by the scope of the appended claims.

1. A composition having a structure as shown in formula I: 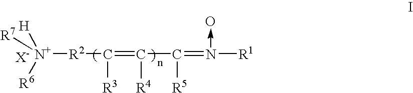
wherein R¹ and R² are independently at each occurrence an aliphatic radical having from 1 to about 10 carbons, a cycloaliphatic radical having from about 3 to about 10 carbons, or an aromatic radical having from about 3 to about 12 carbons; R³, R⁴, and R⁵ are independently at each occurrence a hydrogen atom, an aliphatic radical having from 1 to about 10 carbons, a cycloaliphatic radical having from about 3 to about 10 carbons, or an aromatic radical having from about 3 to about 12 carbons; R⁶ and R⁷ are independently at each occurrence a hydrogen atom or an aliphatic radical having from 1 to about 6 carbons; X is a halogen; and “n” is an integer having a value of from 0 to about 4.

2. The composition as defined in claim 1, wherein R¹ is an aromatic radical having from about 5 to about 12 carbons; R² is an aromatic radical having from about 5 to about 12 carbons; R³, R⁴, and R⁵ are independently at each occurrence a hydrogen atom, an aliphatic radical having from 1 to about 10 carbons, a cycloaliphatic radical having from about 3 to about 10 carbons, or an aromatic radical having from about 3 to about 12 carbons.

3. The composition as defined in claim 1, wherein X is chlorine.

4. The composition as defined in claim 1, wherein X is bromine.

5. The composition as defined in claim 1, wherein X is iodine.

6. The composition as defined in claim 1, wherein R¹ is an aromatic radical having from about 6 to about 10 carbons; R² is an aromatic radical having from about 6 to about 10 carbons; R³, R⁴, and R⁵ are independently at each occurrence a hydrogen atom, an aliphatic radical having from 1 to about 5 carbons, a cycloaliphatic radical having from about 4 to about 8 carbons, or an aromatic radical having from about 6 to about 10 carbons; and “n” is an integer having a value of from 1 to 3.

7. The composition as defined in claim 1, wherein R¹ comprises at least one electron withdrawing substituent having a structure selected from the group consisting of formulae; 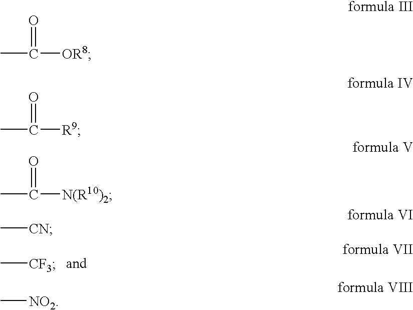
wherein R⁸, R⁹, and R¹⁰ are each independently at each occurrence an aliphatic radical having 1 to 10 carbons, a cycloaliphatic radical having 3 to 10 carbons, and an aromatic radical having 3 to 10 carbons.

8. An article comprising the composition as defined in claim 1.

9. The article as defined in claim 8, wherein the article is a holographic recording medium.

10. A composition having a structure as shown in formula IX.

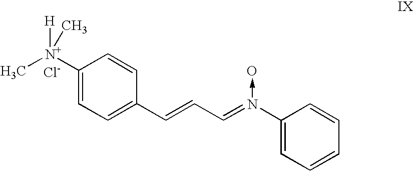

11. An article comprising the composition as defined in claim 10.

12. A composition having a structure as shown in formula X.

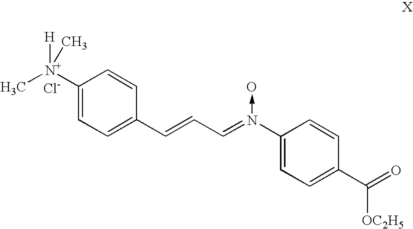

13. An article comprising the composition as defined in claim 12.

14. A method of preparing the composition as defined in claim 1 having a structure as shown in formula I.

15. The method as defined in claim 14, wherein the the composition having a structure as shown in formula I is prepared by protonating a composition having a structure as shown in formula II.

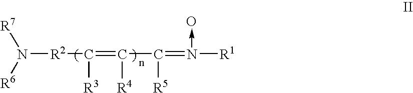

16. A method of preparing the composition as defined in claim 10 having the structure as shown in formula IX.

17. The method as defined in claim 16, wherein the the composition having a structure as shown in formula IX is prepared by protonating a composition having a structure as shown in formula XI.

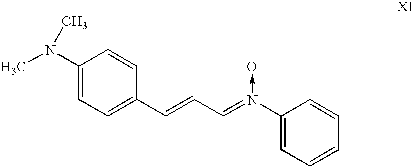

18. A method of preparing the composition as defined in claim 12 having the structure as shown in formula X.

19. The method as defined in claim 12, wherein the the composition having the structure as shown in formula X is prepared by protonating the composition having a structure as shown in formula XII. [chemical structure]
